(19) United States
(12) Patent Application Publication
(10) Pub. No.: US 2023/0390293 A1
(43) Pub. Date: Dec. 7, 2023

(54) PHARMACEUTICAL COMPOSITIONS OF IBRUTINIB

(71) Applicant: CIPLA LIMITED, Mumbai (IN)

(72) Inventors: Geena Malhotra, Mumbai (IN); Jinesh Chauhan, Mumbai (IN); Kishore Kothule, Navi Mumbai (IN); Ravichandra Bhadravathi Vedamurthy, Thane (West) (IN); Anirban Mallik Thakur, Mumbai (IN)

(21) Appl. No.: 18/142,909

(22) Filed: May 3, 2023

Related U.S. Application Data
(62) Division of application No. 16/326,570, filed on Feb. 19, 2019, now abandoned.

(30) Foreign Application Priority Data
Aug. 19, 2016 (IN) 201621028288

(51) Int. Cl. A61K 31/519; A61K 45/06; A61K 9/20; A61P 35/00

(52) U.S. Cl. CPC A61K 31/519; A61K 45/06; A61K 9/2054; A61P 35/00

(57) ABSTRACT

This invention relates to novel pharmaceutical formulations of Bruton's tyrosine kinase (BTK) inhibitor Ibrutinib. This invention also relates to methods of using the Ibrutinib pharmaceutical formulations, alone or in combination with other therapeutic agents, for the treatment of autoimmune diseases or conditions, cancers, including lymphoma, and inflammatory diseases or conditions.

### FIELD OF THE INVENTION

This invention relates to pharmaceutical formulations of Bruton's tyrosine kinase (BTK) inhibitor 1-((R)-3-(4-amino-3-(4-phenoxyphenyl)-1H-pyrazolo[3,4-d]pyrimidin-1-yl)pi-peridin-1-yl)prop-2-en-1-one (Ibrutinib), including methods of using the BTK inhibitor in the treatment of diseases or conditions that would benefit from inhibition of BTK activity.

### BACKGROUND OF THE INVENTION

Ibrutinib is novel BTK inhibitor currently approved and marketed under the brand name IMBRUVICA® 140 mg capsule for treatment of different kinds of cancer, in particular mantle-cell lymphoma (MCL), chronic lymphocytic leukemia (CLL), Small lymphocytic lymphoma (SLL), Waldenstrom's macroglobulinemia (WM) and marginal zone lymphoma (MZL). Imbruvica is also approved for treatment of Chronic graft versus host disease (cGVHD) after failure of one or more lines of systemic therapy.

The recommended dose of IMBRUVICA® is 560 mg (four 140 mg capsules) once daily for MCL and MZL and 420 mg (three 140 mg capsules) once daily for CLL. SLL, WM and cGVHD. Thus, the required dose for MCL and MZL is comprised in four IMBRUVICA® capsules that have to be administered orally once a day and the required dose for CLL, SLL. WM and cGVHD is comprised in three IMBRUVICA® capsules administered orally once a day.

Bruton's tyrosine kinase (BTK), a member of the Tee family of non-receptor tyrosine kinases, is a key signalling enzyme expressed in all hematopoietic cells types except T lymphocytes and natural killer cells. BTK plays an essential role in the B-cell signalling pathway linking cell surface B-cell receptor (BCR) stimulation to downstream intracellular responses.

BTK is a key regulator of B-cell development, activation, signalling, and survival. In addition, BTK plays a role in a number of other hematopoietic cell signalling pathways, e.g., Toll like receptor (TLR) and cytokine receptor-mediated TNF-α production in macrophages, IgE receptor signalling in Mast cells, inhibition of Fas/APO-1 apoptotic signalling in B-lineage lymphoid cells, and collagen-stimulated platelet aggregation

U.S. Pat. No. 8,008,309 discloses the chemical synthesis of Ibrutinib and its analogues. U.S. Pat. No. 9,296,753 discloses various polymorphic forms of Ibrutinib such as Form A, B, C, D, E and F, pharmaceutical compositions prepared using said polymorphs and uses thereof. US patent application no. 20160038496 discloses novel solid dispersed formulations of Ibrutinib wherein Ibrutinib may be spray-dried prior to preparing said solid dispersed formulations.

Ibrutinib is a weak base which is poorly soluble in water and shows an electrostatic behaviour with a strong tendency to agglomerate. This results in a low bulk density and challenges to get a homogeneous distribution of the active ingredient within solid oral dosage forms, like immediate release capsules and tablets.

An object of the present invention is thus to provide pharmaceutical compositions of Ibrutinib having a homogeneous distribution of the active ingredient. A further object of the present invention is to provide pharmaceutical compositions of Ibrutinib having a high bioavailability.

A yet another object of the present invention is to provide pharmaceutical compositions of Ibrutinib having fast disintegration after dispersion in water.

A still another object of the present invention is to provide pharmaceutical compositions of Ibrutinib having fast dissolution and high bioavailability.

A yet another object of the present invention is to provide pharmaceutical compositions of Ibrutinib for use in the treatment of cancer having reduced pill burden and improved patient compliance.

A yet another object of the present invention is to provide pharmaceutical compositions of Ibrutinib for use in the treatment of cancer having lower dosages or strengths of the active ingredient.

A further object of the present invention is to provide pharmaceutical compositions of Ibrutinib for use in the treatment of cancer that causes reduced adverse events and enhances patient safety.

### BRIEF DESCRIPTION OF THE DRAWINGS

FIG. 1 shows plasma concentration-time profiles of Ibrutinib following oral administration of three Imbruvica® capsules and one capsule containing the test formulation (Test formulation-1) to fasted male beagle dogs.

FIG. 2 shows plasma concentration-time profiles of Ibrutinib following oral administration of one capsule each of RLD and test formulations to fasted male beagle dogs.

FIG. 3 shows plasma concentration-time profiles of Ibrutinib following oral administration of one capsule each of RLD and test formulations to fasted male beagle dogs.

FIG. 4 shows the in-vitro dissolution rate of RLD and test formulations in 3% Tween 20 with pH 6.8 phosphate buffer, 900 mL, USP type II apparatus at paddle speed of 75 rpm.

FIG. 5 shows the in-vitro dissolution rate of RLD and test formulations at pH of 1.2 buffer, 900 mL, USP type II apparatus at a paddle speed of 50 rpm.

### DETAILED DESCRIPTION OF THE INVENTION

It has been found that Ibrutinib compositions of the present invention provide high bioavailability of Ibrutinib. In another aspect of the invention Ibrutinib compositions of the present invention provide high bioavailability even at less than lower dosage amounts of Ibrutinib.

The number of units to be taken at a time and the multiple drug regimens of the dosage forms which are to be administered at the same time pose issues during the therapy specifically in context of the patient compliance. Patient compliance in such a regimen can be addressed by decreasing the number of tablets or capsules administered as well as the type of dosage forms that are administered, with due consideration to the bioavailability of the administered drug. The bioavailability of the drug cannot be compromised to meet patient compliance.

The above criteria could be met by formulating a composition with a reduced or low dose such that it exhibits similar or increased bioavailability. Hence, Ibrutinib as an active pharmaceutical agent used for treating lymphoma or leukaemia, would be, preferred in a low dose oral composition provided in such a dosage form which exhibits desired therapeutic effect and at the same time ensures patient compliance.

Dose of Ibrutinib will depend on the use such as therapeutic, prophylactic, or maintenance. Dose of Ibrutinib will also depend on severity and progression of the disease or condition such as cancers (Relapsed or refractory), previous therapy, the patient's health status, weight, and response to the drugs, and the judgment of the physician.

The present invention provides a low dose pharmaceutical composition comprising Ibrutinib which would ensure patient compliance due to simplification of therapy, ease of administration, an acceptable dosing regimen and bioavailability. The high bioavailability of the compositions of the present invention may be attributable to the rapid dissolution thereby allowing faster and higher drug absorption. While not wishing to be bound by any theory, it is believed that substantially enhanced dissolution of compositions of the present invention may be responsible for the rapid absorption and consequent higher bioavailability of Ibrutinib.

The dose which can be lower than the usual or the conventional dose, required to produce equal or higher therapeutic effect, may also reduce the side effects thereby leading to limit the risk to the patient.

The term “Ibrutinib” is used in broad sense to include not only “Ibrutinib” per se but also its pharmaceutically acceptable derivatives thereof. Suitable pharmaceutically acceptable derivatives include pharmaceutically acceptable salts, solvates, hydrates, anhydrates, enantiomers, esters, isomers, polymorphs, prodrugs, tautomers, complexes etc.

The term “low dose” as used herein refers to a therapeutically effective dose of Ibrutinib, which dose is less than the usual or the conventional dose required to produce equal or higher therapeutic effect.

Ibrutinib may be administered at least once, twice or thrice a day in the dosing range from about 10 mg to about 1200 mg. Preferably, Ibrutinib may be administered at least once, twice or thrice a day in the dosing range from about 10 mg to about 800 mg. Preferably, Ibrutinib may be administered at least once, twice or thrice a day in the dosing range from about 10 mg to about 600 mg. Preferably Ibrutinib may be administered at least once, twice or thrice a day in the dosing range from about 10 mg to about 560 mg. Preferably Ibrutinib may be administered at least once, twice or thrice a day in the dosing range from about 10 mg to about 420 mg. Preferably Ibrutinib may be administered at least once, twice or thrice a day in the dosing range from about 10 mg to about 280 mg. Preferably Ibrutinib may be administered at least once, twice or thrice a day in the dosing range from about 10 mg to about 200 mg. Preferably Ibrutinib may be administered at least once, twice or thrice a day in the dosing range from about 10 mg to about 140 mg. Preferably Ibrutinib may be administered at least once, twice or thrice a day in the dosing range from about 10 mg to about 100 mg.

The preferred dosing range for Ibrutinib may be from about 100 mg to about 250 mg once daily. More preferably the dose of Ibrutinib or its pharmaceutically acceptable salt may be from about 125 mg to about 250 mg once daily. More preferably the dose of Ibrutinib or its pharmaceutically acceptable salt may be from about 140 mg to about 250 mg once daily. More preferably the dose of Ibrutinib or its pharmaceutically acceptable salt may be from about 150 mg to about 250 mg once daily. More preferably the dose of Ibrutinib or its pharmaceutically acceptable salt may be from about 175 mg to about 250 mg once daily. More preferably the dose of Ibrutinib or its pharmaceutically acceptable salt may be from about 200 mg to about 250 mg once daily. Even more preferably the dose of Ibrutinib may be from about 225 mg to about 250 mg once daily.

In even more preferred embodiments dose of Ibrutinib may be about 100, 110, 120, 130, 140, 150, 160, 170.180, 190, 200, 210, 220, 230, 240, 250 mg once daily.

In more preferred embodiments, the dose of Ibrutinib may be from about 1 mg/kg/day to about 13 mg/kg/day. In more preferred embodiments, the dose of Ibrutinib may be from about 2.5 mg/kg/day to about 10 mg/kg/day. In more preferred embodiments, the dose of Ibrutinib may be from 2.5 mg/kg/day to about 6 mg/kg/day. In some embodiments, the dose of Ibrutinib may be from about 2.5 mg/kg/day to about 4 mg/kg/day. In even more preferred embodiment, the dose of Ibrutinib is about 2.5 mg/kg/day.

Preferably, Ibrutinib is administered until disease progression, unacceptable toxicity, or individual choice. In some embodiments, Ibrutinib is administered at least once daily until disease progression, unacceptable toxicity, or individual choice. In some embodiments, Ibrutinib is administered every other day until disease progression, unacceptable toxicity, or individual choice.

Ibrutinib or its pharmaceutically acceptable salts may be provided in the form of a pharmaceutical composition such as but not limited to, unit dosage forms including tablets, disintegrating tablets, dispersible tablets, granules, capsules (filled with solid dispersion, solid solution, powders, pellets, beads, mini-tablets, pills, micro-pellets, small tablet units, multiple unit pellet systems (MUPS)), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Optimization of the particle size of Ibrutinib can provide a lower maximum concentration (cₘₐₓ) of Ibrutinib thereby reducing side effects, reducing or nullifying the food effect and can help increase bioavailability of Ibrutinib thereby enabling a reduction in daily dose.

In one embodiment. Ibrutinib may have an average particle size of less than 200 micron. In one embodiment, Ibrutinib may have an average particle size of less than 150 micron. In one embodiment, Ibrutinib may have an average particle size of less than 100 micron. In one embodiment, Ibrutinib may have an average particle size of less than 40 micron. More preferably the average particle size is less than 20 micron. Even more preferably the average particle size is less than 10 micron. Even more preferably the average particle size is less than 5 micron. Even more preferably the average particle size is less than 2 micron.

In one embodiment, Ibrutinib may be present in the form of nanoparticles which may have an average particle size of less than 2000 nm.

In a further aspect there are provided pharmaceutical compositions, which include Ibrutinib and at least one additional ingredient selected from pharmaceutically acceptable carriers, diluents and excipients. In some embodiments, the pharmaceutical composition comprises Ibrutinib in amorphous form. In some embodiments, the pharmaceutical composition comprises Ibrutinib as polymorphic form A. In some embodiments, the pharmaceutical composition comprises Ibrutinib polymorphic form B. In some embodiments, the pharmaceutical composition comprises Ibrutinib polymorphic form C. In some embodiments, the pharmaceutical composition comprises Ibrutinib polymorphic form D. In some embodiments, the pharmaceutical composition comprises Ibrutinib polymorphic form E. In some embodiments, the pharmaceutical composition comprises Ibrutinib polymorphic form F.

In more preferred embodiments, the pharmaceutical composition comprises amorphous Ibrutinib. Preferably the pharmaceutical composition is prepared using Ibrutinib polymorphic form C. Even more, preferably the pharmaceutical composition is prepared using amorphous Ibrutinib. Most preferably the pharmaceutical composition is prepared using Ibrutinib polymorphic form C. Various polymorphic forms of Ibrutinib are disclosed in WO 2013 184572A1 which is incorporated herein by reference.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

Suitable disintegrants or super disintegrants may comprise agar-agar, calcium carbonate, microcrystalline cellulose, crospovidone, povidone, polacrilin potassium, sodium starch glycolate, potato or tapioca starch, other starches, pre-gelatinized starch, clays, alginic acid, alginates such as sodium alginate other algins, other celluloses, gums, ion-exchange resins, magnesium aluminium silicate, sodium dodecyl sulfate, sodium carboxymethyl cellulose, croscarmellose sodium, polyvinyl pyrollidone, cross-linked PVP, carboxymethyl cellulose calcium, cross-linked sodium carboxymethyl cellulose, docusate sodium, guar gum, low-substituted HPC, polacrilin potassium, poloxamer, povidone, sodium glycine carbonate and sodium lauryl sulfate or mixtures thereof.

The amount of disintegrant in the low dose pharmaceutical compositions may range from about 3% w/w to about 30% w/w, of the total weight of the composition.

Suitable binders for use, in the low dose pharmaceutical composition of the present invention may comprise one or more, but not limited to polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethyl cellulose, dextrin, gelatin, gum arabic, guar gum, tragacanth, sodium alginate, or mixtures thereof or any other suitable binder.

The amount of binder in the low dose pharmaceutical compositions may range from about 5% w/w to about 20% w/w, of the total weight of the composition.

Suitable carriers, diluents or fillers for use, in the low dose pharmaceutical composition of the present invention may comprise one or more, but not limited to lactose (for example, spray-dried lactose, a-lactose, β-lactose) lactose, lactose monohydrate, available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropyl cellulose, L-hydroxypropyl cellulose (low substituted), hydroxypropyl methylcellulose (HPMC), methylcellulose polymers (such as, for example, Methocel A, Methocel A4C, Methocel A15C, Methocel A4M), hydroxy ethyl cellulose, sodium carboxymethyl cellulose, carboxymethylene, carboxymethyl hydroxyethyl cellulose and other cellulose derivatives, starches or modified starches (including potato starch, corn starch, maize starch and rice starch) or mixtures thereof.

The amount of carriers, diluents or fillers in the low dose pharmaceutical compositions may range from about 15% w/w to about 60% w/w.

Glidants, anti-adherents and lubricants may also be incorporated in the low dose pharmaceutical composition of the present invention, which may comprise one or more of, but not limited to stearic acid and pharmaceutically acceptable salts or esters thereof (for example, magnesium stearate, calcium stearate, zinc stearate, sodium stearyl fumarate or other metallic stearate), talc, waxes (for example, microcrystalline waxes) and glycerides, mineral oil, light mineral oil, PEG, silica acid or a derivative or salt thereof (for example, silicates, silicon dioxide, colloidal silicon dioxide and polymers thereof, crospovidone, magnesium aluminosilicate and/or magnesium aluminometasilicate), silicified microcrystalline cellulose such as Prosolv SMCC 90 (silicified microcrystalline cellulose composed of 98% microcrystalline cellulose and 2% colloidal silicon dioxide), sucrose ester of fatty acids, hydrogenated vegetable oils (for example, hydrogenated castor oil, peanut oil, cottonseed oil, sunflower oil, sesame oil, olive oil, corn oil and soybean oil), glycerin, sorbitol, mannitol, other glycols, sodium lauryl sulfate, talc, long chain fatty acids and their salts, ethyl oleate, ethyl laurate, agar, syloid silica gel (a coagulated aerosol of synthetic silica (Evonik Degussa Co., Piano, Tex. USA), a pyrogenic silicon dioxide (CAB-O-SIL, Cabot Co., Boston, Mass. USA), or mixtures thereof.

The amount of glidants, anti-adherents and lubricants in the low dose pharmaceutical compositions may range from about 0.1% w/w to about 5% w/w.

Suitable anti-caking additives may comprise, but are not limited to, calcium silicate, magnesium silicate, silicon dioxide, colloidal silicon dioxide, talc, or mixtures thereof.

Suitable antioxidants may comprise, but are not limited to, tocopherols, ascorbic acid, sodium pyrosulfite, butylhydroxytoluene, butylated hydroxyanisole, edetic acid, and edetate salts, or mixtures thereof.

Suitable texture enhancers may comprise, but are not limited to, pectin, polyethylene oxide, and carrageenan, or mixtures thereof.

Surface stabilizers, according to the present invention, are surfactants that are capable of stabilizing the increased surfaced charge drug. Suitable amphoteric, non-ionic, cationic or anionic surfactants may be included in the low dose pharmaceutical composition of the present invention.

According to the present invention, surfactants may comprise of one or more of, but not limited to Polysorbates, Sodium dodecyl sulfate (sodium lauryl sulfate), Lauryl dimethyl amine oxide, Docusate sodium. Cetyltrimethyl ammonium bromide (CTAB) Polyethoxylated alcohols, Polyoxyethylenesorbitan, Octoxynol, N, N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, NonylphenolethoxylateCyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates. Sulphated natural oils & fats, Sulphated esters, Sulphatedalkanolamides, Alkylphenols, ethoxylated& sulphated, Ethoxylated aliphatic alcohol, polyoxyethylene surfactants, carboxylic esters Polyethylene glycol esters, Anhydrosorbitol ester & it's ethoylated derivatives, Glycol esters of fatty acids, Carboxylic amides. Monoalkanolamine condensates, Polyoxyethylene fatty acid amides. Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt, Phosal 53 MCT, Tweens, Polyoxyethylene (20) sorbitantrioleate (Tween 85), Oleoylmacrogolglycerides (Labrafil M1944CS), Linoleoylmacrogolglycerides (Labrafil M2125CS), PG monolaurate (Lauroglycol 90), D-alpha-tocopheryl PEG 1000 succinate (Vitamin E TPGS), Polyoxyl 35 castor oil (Cremophor EL, Cremophor ELP), Polvoxyl 40 hydrogenated castor oil (Cremophor RH 40, Cremophor RH 60), Lauroylmacrogolglycerides (Gelucire 44/14, Gelucire 50/13), Lauroyl macrogol-32 glycerides. Lauroyl polyoxyl-32 glycerides, Lauroylpolyoxylglycerides, Caprylocaproylmacrogol glycerides (Labrasol), Polyoxyethylene (20) sorbitanmonooleate, (Polysorbate 80/Tween 80). Polyoxyethylene (20) sorbitanmonolaurate (Polysorbate 20/Tween 20), polyglycerol (polyglyceryloleate: Plural™ Oleique CC497) propylene glycol (propylene glycol monocaprylate: Capryol™ 90, propylene glycol monolaurate; Lauroglycol 90), polyoxyethylene glycols (PEG-8 stearate: Mirj 45, PEG-40 stearate: Mirj 52, PEG-15 hydroxystearate: Solutol® HS15), sorbitan or monoanhydrosorbitol (sorbitanmonooleate: Span® 80, sucrose (sucrose monopalmitate: Surfhope® D-1616), Lutrol E 300, Transcutol HP, Transcutol P, Soyabean oil, Labrafac PG, Milyol 840, Pluronic L44, Pluronic L64, Polaxamer 188, and the like or mixtures thereof.

The amount of surfactant in the pharmaceutical compositions may range from about 2% w/w to about 10% w/w, of the total weight of the composition. Compositions devoid of surfactants are also be envisaged under the ambit of the present invention. Preferably the composition is devoid of sodium dodecyl sulfate (sodium lauryl sulfate).

Viscosity modifying agents are excipients that are capable of stabilizing the formulation by increasing the viscosity of the formulation and thus preventing physical interaction of nanoparticles under the operating conditions employed.

The amount of viscosity modifying agents in the pharmaceutical compositions may range from about 4% w/w to about 20% w/w, of the total weight of the composition.

According to the present invention, viscosity modifying agents may comprise one or more of, but not limited to derivatives of sugars, such as lactose, sucrose, saccharose, hydrolyzed starch (maltodextrin) or mixtures thereof.

Polymers or polymers blends, according to the present invention, may comprise one or more of hydrophilic polymers, but not limited to cellulose derivates like hydroxypropyl cellulose, hydroxymethyl cellulose, hydroxypropyl methylcellulose, hydroxypropylcellulose acetate-succinate, hydroxypropyl methyl cellulose phthalate, or cellulose acetate phthalate, methylcellulose polymers hydroxyethyl cellulose, sodium carboxymethyl cellulose, carboxymethylene and carboxymethyl hydroxyethyl cellulose: acrylics like acrylic acid, acrylamide, and maleic anhydride polymers, polyvinyl caprolactam-polyvinyl acetate-polyethylene glycol graft copolymer (Soluplus®), polyvinylpyrrolidone/vinyl acetate co-polymer, acacia, gum tragacanth, locust bean gum, guar gum, or karaya gum, agar, pectin, carrageenan, gelatin, casein, zein and alginates, carboxypolymethylene, bentonite, magnesium aluminum silicate, polysaccharides, modified starch derivatives and copolymers or mixtures thereof. In one embodiment, the polymer is hydroxypropyl methyl cellulose acetate. In yet another embodiment, the polymer is hydroxypropyl methyl cellulose acetate succinate (HPMCAS). In yet another embodiment the polymer is polyvinyl caprolactam-polyvinyl acetate-polyethylene glycol graft copolymer (Soluplus®). In yet another embodiment the polymer is polyvinylpyrrolidone/vinyl acetate co-polymer, optionally in combination with polyvinyl caprolactam-polyvinyl acetate-polyethylene glycol graft copolymer (Soluplus®).

The amount of polymers or polymers blends in the pharmaceutical compositions may range from about 2% w/w to about 75% w/w, of the total weight of the composition.

Suitable channeling agents for use in compositions of the invention, may comprise one or more, but are not limited to sodium chloride, sugars, polyols and the like or mixtures thereof.

Preferably, the channeling agents may be present in an amount ranging from about 0.5% to about 10% by weight of the composition.

The low dose pharmaceutical composition, according to the present invention, may also optionally be coated, but not limited to seal coating, enteric coating, film coating or a combination thereof.

According to an embodiment of the present invention, low dose pharmaceutical composition may be film coated, seal coated or enteric coated with, but not limited to, eudragit L30 D55, other grades of Eudragit, hydroxypropyl methyl cellulose pthalate, cellulose acetate phthalate, colour mix systems (such as Opadry colour mix systems), Aqueous Acrylic Enteric System (such as Acryl-EZE®) and Kollicoat) Protect.

Preferably, the low dose pharmaceutical composition, according to the present invention, may be film coated.

According to the present invention, the seal coat comprises film forming polymeric materials, such as but not limited to, hydroxypropylmethyl cellulose, hydroxypropyl cellulose, polyvinyl pyrrolidone, methylcellulose, carboxymethyl cellulose, hypromellose, acacia, gelatin to increase adherence and coherence of the seal coat.

The amount of seal coating system in the low dose pharmaceutical compositions may range from about 1% w/w to about 3% w/w, of the total weight of the composition.

According to the present invention, pharmaceutically acceptable opacifier for use in the low dose pharmaceutical composition of the present invention may comprise one or more, but is not limited to titanium dioxide.

The low dose pharmaceutical composition, according to the present invention, may exhibit bioavailability to an extent to produce the desired pharmacological effects along with reduced side effects after dosing in a subject.

The present invention also provides a method of treating mantle cell lymphoma by administering a low dose pharmaceutical composition comprising Ibrutinib.

The present invention also provides a method of treating relapsed or refractory mantle cell lymphoma by administering a low dose pharmaceutical composition comprising Ibrutinib.

The present invention also provides a method of treating chronic lymphocytic leukaemia by administering a low dose pharmaceutical composition comprising Ibrutinib.

The present invention also provides a method of treating chronic lymphocytic leukaemia with 17p deletion by administering a low dose pharmaceutical composition comprising Ibrutinib.

The present invention also provides a method of treating small lymphocytic lymphoma by administering a low dose pharmaceutical composition comprising Ibrutinib.

The present invention also provides a method of treating small lymphocytic lymphoma with 17p deletion by administering a low dose pharmaceutical composition comprising Ibrutinib.

The present invention also provides a method of treating Waldenstrom's macroglobulinemia by administering a low dose pharmaceutical composition comprising Ibrutinib.

The present invention also provides a method of treating Marginal zone lymphoma by administering a low dose pharmaceutical composition comprising Ibrutinib.

The present invention also provides the use of the low dose pharmaceutical composition comprising Ibrutinib for treating mantle cell lymphoma.

The present invention also provides the use of the low dose pharmaceutical composition comprising Ibrutinib for treating chronic lymphocytic leukaemia.

The low dose pharmaceutical composition of the present invention, may further comprise at least one additional active ingredient such as, but not limited to, MEK inhibitor, topoisomerase inhibitor, EGFR inhibitor, anti-CTLA4 antibody, DLL4 antagonist, anti-HMW-MAA antibody, peginterferon alfa-2a, dihydroorotate dehydrogenase inhibitor, AKT inhibitor compounds, tyrosine kinase inhibitor, inhibitor of CDK4, PI3K beta inhibitor, MAPK pathway inhibitor, interleukin-2, c-Met antagonists, Hsp90 inhibitors, Wnt pathway inhibitors, pyruvate dehydrogenase kinase inhibitors, ERK pathway inhibitors, anti-ErbB3 antibody, MDM2 inhibitor.

For combination therapies described herein, dosages of the co-administered compounds vary depending on the type of co-drug employed, on the specific drug employed, on the disease or condition being treated. The individual drugs to be used in combinations are administered either sequentially or simultaneously in separate or combined pharmaceutical compositions.

The combinations may be conveniently presented for use for treatment of diseases and disorders in the form of a pharmaceutical compositions together with a pharmaceutically acceptable diluent(s) or carrier(s).

In certain embodiments, is a method for treating a cancer in an individual in need thereof, comprising: administering to the individual an amount of Ibrutinib. In some embodiments, the method further comprises administering a second cancer treatment drug.

In certain embodiments, the combinations of Ibrutinib with active ingredients described herein may be used for treatment of B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

In order that this invention be more fully understood, the following preparative and testing methods and examples are set forth. These examples are for the purpose of illustration only and are not to be construed as limiting the scope of the invention in any way.

### EXAMPLES

### Example 1

Ibrutinib Capsule Prepared by Hot Melt Extrusion

| | | | |
|---|---|---|---|
| Sr. | | | Proposed % |
| No. | Ingredients | Purpose | Concentration |

Dry mix

| | | | |
|---|---|---|---|
| 1. | Ibrutinib (form C) | Active | 25-50% |
| 2. | Polymer for HME process | Polymer | 25-75% |
| | (Co povidone or other | | |
| | suitable polymer) | | |
| 3. | Plasticizer (Span 20 or other | Plasticizer | 2.5-7.5% |
| | suitable plasticizer) | | |
| 4. | Colloidal silicon dioxide | Glidant | 0.1-1.0% |

Lubrication

| | | | |
|---|---|---|---|
| 5. | Magnesium stearate | Lubricant | 0.1-1.0% |

| | |
|---|---|
| Total | 100.000 |

Capsule filling

| | | | |
|---|---|---|---|
| 6. | Size 0 hard gelatin capsule | — | — |

Process:

Ibrutinib was mixed with a polymer premix containing co-povidone and span 20. The mix is then extruded using a hot melt extruder, sized by co-milling, lubricated with colloidal silicon dioxide in an octagonal blender and filled in hard gelatine capsules.

### Example 2

Ibrutinib Capsule Prepared by Dry Granulation

| | | | | |
|---|---|---|---|---|
| Sr. | | | % | |
| No. | Ingredients | Purpose | Concentration | Mg/cap |

Dry mix

| | | | | |
|---|---|---|---|---|
| 1. | Ibrutinib (Form C) | Active | 42.42 | 140.0 |
| 2. | Microcrystalline Cellulose | Diluent | 45.22 | 149.25 |
| 3. | Croscarmellose sodium | Disintegrant | 7.00 | 23.10 |
| 4. | Magnesium stearate | Lubricant | 0.25 | 0.825 |

Lubrication

| | | | | |
|---|---|---|---|---|
| 5. | Microcrystalline Cellulose | Diluent | 4.849 | 16.00 |
| | Magnesium stearate | Lubricant | 0.25 | 0.825 |

| | | |
|---|---|---|
| Total | 100.0 | 330.0 |

Process:

Ibrutinib, microcrystalline cellulose, croscarmellose sodium and magnesium stearate were mixed to prepare a uniform blend.

The powder blend is then compacted using roller compaction process.

The granule particles are co-milled to obtain a uniform blend.

The granules are lubricated with magnesium stearate and microcrystalline cellulose in an octagonal blender and filled in size 0 capsules.

Study I:

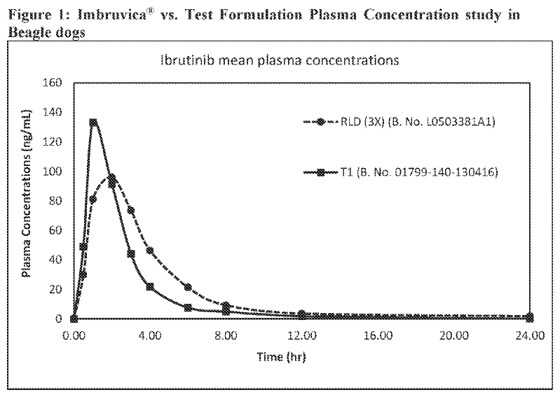

Pharmacokinetic parameters of composition prepared in example 2 (Test-1/T1) were compared with that of Imbruvica® capsule. One capsule of test-1 and three capsules of Imbruvica were administered orally to fasted male beagle dogs. The dogs were dosed in two periods in a unique cross over study design (TABLE 1). Five animals per group were dosed in each period and a minimum five days of washout period was given between the doses (FIG. 1).

TABLE 1

| | | | | Dog ID No. | |
|---|---|---|---|---|---|
| Group | Treatment | Batch No. | Dose | Period 1 | Period 2 |
| G1 | RLD (3X): Ibrutinib 140 mg | L0503381A1 | 3 capsule per dog | 1-5 | 6-10 |
| | capsule (IMBRUVICA ®) | | (420 mg/dog) | | |
| G2 | Test-1 (IX): Ibrutinib 140 mg | 01799-140- | 1 capsule per dog | 6-10 | 1-5 |
| | capsule (Cipla) | 130416 | (140 mg/dog) | | |
| G3 | RLD (IX): Ibrutinib 140 mg | L0503381A1 | 1 capsule per dog | 11-15 | 16-20 |
| | capsule (IMBRUVICA ®) | | (140 mg/dog) | | |
| G4 | Test-2 (IX): Ibrutinib 140 mg | 01799-140- | 1 capsule per dog | | |
| | capsule | 310516A | (140 mg/dog) | 16-20 | 11-15 |

Results:

| | RLD | |
|---|---|---|
| PK Parameters | (3X) | T1 |
| AUCₗₐₛₜ (h*ng/ml) | 424 | 333 |
| AUC INF_obs (hr*ng/mL) | 424 | 339 |
| AUC₀₋₁₂ (h*ng/mL) | 393 | 326 |
| Cₘₐₓ (ng/mL) | 133 | 153 |
| Tₘₐₓ (h)-Median | 2.00 | 1.00 |
| t_{1/2} (h) | 5.66 | 3.29 |
| Vz_F_obs (L) | 20027 | 3345 |

### Example 3

| | | | | |
|---|---|---|---|---|
| Sr. | | | % | |
| No. | Ingredients | Purpose | Concentration | Mg/cap |

Dry mix

| | | | | |
|---|---|---|---|---|
| 1. | Ibrutinib (form A) | Active | 35.0 | 140.0 |
| 2. | Microcrystalline Cellulose | Diluent | 55.2 | 221.0 |
| 3. | Croscarmellose sodium | Disintegrant | 5.1 | 20.5 |
| 4. | Sodium lauryl sulfate | Surfactant | 3.5 | 14.0 |
| 5. | Magnesium stearate | Lubricant | 1.1 | 4.5 |

| | | |
|---|---|---|
| Total | 100.0 | 400.0 |

Process:

Ibrutinib, microcrystalline cellulose, croscarmel lose sodium, sodium lauryl sulfate and magnesium stearate were mixed to prepare a uniform blend.

The powder blend is then compacted using roller compaction process.

The granule particles are co-milled to obtain a uniform blend.

The granules are lubricated with Magnesium stearate in an octagonal blender and filled in size 0 capsules.

### Example 4

The following compositions were also prepared by using process as disclosed in example 2 above:

| | | 01799- | 01799- | 01799- | 01799- | 01799- | 01799- | 01799- |
|---|---|---|---|---|---|---|---|---|
| s. | | 140- | 140- | 140- | 140- | 140- | 140- | 140- |
| No. | Ingredients | 020116 | 030116 | 040116 | 120416 | 130416 | 140416 | 150416 |
| | API | A mor- | Amor | Crystalline | Crystalline | Crystal line | Amor- | Amor- |
| | Form | phous I | phous-II | (Form A) | (Form C) | (Form C) | phousII | phous II |
| 01 | Ibrutinib | 140.00 | 140.00 | 140.00 | 140.00 | 140.00 | 140.00 | 140.00 |
| 02 | MCC | 135.40 | 135.40 | 135.40 | 135.40 | 149.25 | 135.40 | 149.25 |
| | (Avicel | | | | | | | |
| | PH 102) | | | | | | | |
| 03 | CCS | 23.10 | 23.10 | 23.10 | 23.10 | 23.10 | 23.10 | 23.10 |
| | (Ac-Di- | | | | | | | |
| | Sol) | | | | | | | |
| 04 | SLS | 13.85 | 13.85 | 13.85 | 13.85 | — | 13.85 | — |
| | (Kolliphor | | | | | | | |
| | SLS | | | | | | | |
| | fine) | | | | | | | |
| 05 | Magnesium | 0.825 | 0.825 | 0.825 | 0.825 | 0.825 | 0.825 | 0.825 |
| | stearate | | | | | | | |
| | After | | | | | | | |
| | compaction | | | | | | | |
| 06 | MCC | 16.00 | 16.00 | 16.00 | 16.00 | 16.00 | 16.00 | 16.00 |
| | (Avicel | | | | | | | |
| | PH 102) | | | | | | | |
| 07 | Magnesium | 0.825 | 0.825 | 0.825 | 0.825 | 0.825 | 0.825 | 0.825 |
| | stearate | | | | | | | |
| | Total | 330.00 | 330.00 | 330.00 | 330.00 | 330.00 | 330.00 | 330.00 |

### Example 5

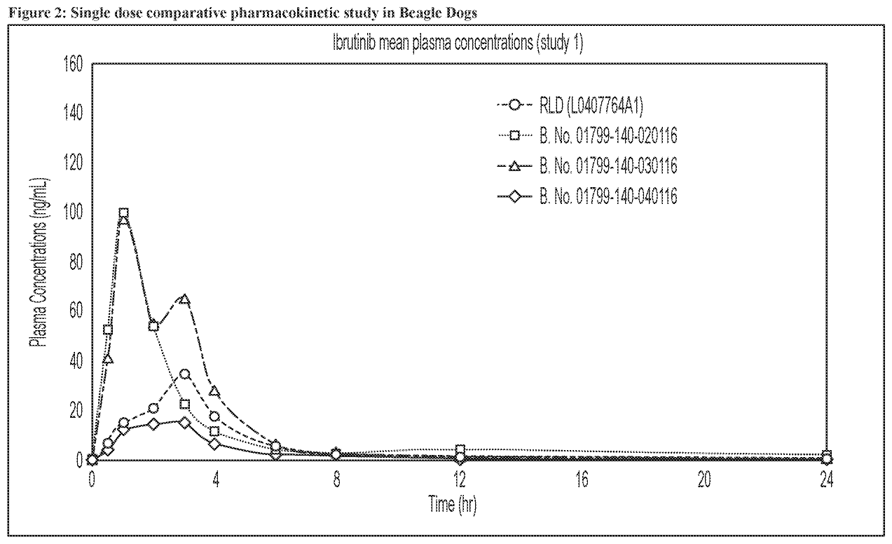

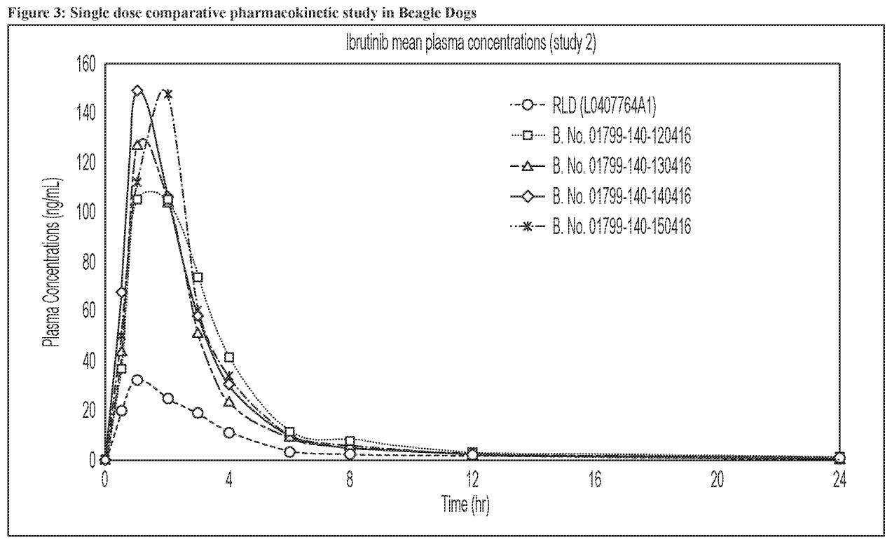

Pharmacokinetic parameters of compositions prepared in example 4 were compared w it h that of Imbruvica⁺ capsule (RLD-L0407764A 1). Table 2 and Table 3 illustrate the pharmacokinetic parameters of RLD compared to the compositions of the invention. One capsule of either test or RLD was administered orally to fasted male beagle dogs. The dogs were dosed in two periods in a unique cross over study design. Eight and six animals per group in study 1 (FIG. 2) and study 2 (FIG. 3) respectively were dosed in each period and a minimum of five days of washout period was given between the doses.

TABLE 2
Comparative Pharmacokinetic study in Beagle Dogs

| Pharmacokinetic | Treatment group (n = 16) | | | |
|---|---|---|---|---|
| Parameters | | 01799-140- | 01799-140- | 01799-140- |
| (arithmetic mean) | L0407764A1 | 020116 | 030116 | 040116 |
| AUCₗₐₛₜ | 117.6 | 247.4 | 277.1 | 58.5 |
| (h*ng/mL) | | | | |
| Cₘₐₓ (ng/mL) | 46.7 | 107.3 | 143.9 | 23.3 |

TABLE 3
Comparative Pharmacokinetic study in Beagle Dogs

| Pharmacokinetic | Treatment group (n = 12) | | | | |
|---|---|---|---|---|---|
| Parameters | | 01799-140- | 01799-140- | 01799-140- | 01799-140- |
| (arithmetic mean) | L0407764A1 | 120416 | 130416 | 140416 | 150416 |
| AUCₗₐₛₜ | 75 | 367 | 266 | 357 | 352 |
| (h*ng/mL) | | | | | |
| Cₘₐₓ (ng/mL) | 26 | 126 | 109 | 147 | 143 |

### Example 6

In-vitro dissolution tests for Ibrutinib compositions prepared in example 4 were performed in 3% Tween 20 with pH 6.8 phosphate buffer, 900 mL, USP type II apparatus at paddle speed of 75 rpm (Table 4) and at pH of 1.2 buffer, 900 mL, USP type II apparatus at a paddle speed of 50 rpm (Table 5).

TABLE 4

| | Media | | | | | | | | | | | | | | | |
|---|---|---|---|---|---|---|---|---|---|---|---|---|---|---|---|---|
| | 3% Tween 20 in pH 6.8 phosphate buffer, 900 mL, USP type II (paddle), 75 rpm | | | | | | | | | | | | | | | |
| | Batch No. | | | | | | | | | | | | | | | |
| | | | 01799-140- | | 01799-140- | | 01799-140- | | 01799-140- | | 01799-140- | | 01799-140- | | 01799-140- | |
| | | | 020116 | | 030116 | | 040116 | | 120416 | | 130416 | | 140416 | | 150416 | |
| | L0407764A1 | | Amorphous- | | Amorphous- | | Crystalline | | Crystalline | | Crystalline | | Amorphous | | Amorphous | |
| Time | RLD | | I* | | II* | | Form A | | form C | | form C | | II | | II | |
| Points | Mean | % RSD | Mean | % RSD | Mean | % RSD | Mean | % RSD | Mean | % RSD | Mean | % RSD | Mean | % RSD | Mean | % RSD |
| 0 | 0 | 0 | 0 | 0 | 0 | 0 | 0 | 0 | 0 | 0 | 0 | 0 | 0 | 0 | 0 | 0 |
| 5 | 42 | 10.87 | 27 | 4.94 | 34 | 6.96 | 56 | 14.17 | 60 | 8.51 | 76 | 5.1 | 30 | 7.5 | 15 | 14.54 |
| 10 | 65 | 3.32 | 34 | 4.57 | 54 | 4.89 | 74 | 8.56 | 77 | 11.09 | 92 | 4.56 | 44 | 7.41 | 24 | 18.34 |
| 15 | 73 | 2.57 | 39 | 4.87 | 62 | 4.62 | 79 | 6.49 | 87 | 11.35 | 96 | 4.01 | 51 | 5.92 | 29 | 18.89 |
| 20 | 78 | 2.3 | 42 | 4.71 | 67 | 4.56 | 82 | 4.71 | 92 | 9.26 | 97 | 4.12 | 55 | 5.04 | 32 | 19.46 |
| 30 | 83 | 1.94 | 47 | 4.5 | 73 | 4.18 | 85 | 3.1 | 97 | 4.65 | 98 | 3.35 | 62 | 4.05 | 36 | 19.4 |
| 45 | 88 | 1.63 | 51 | 4.47 | 79 | 3.58 | 90 | 1.88 | 99 | 3.62 | 98 | 3.21 | 66 | 3.45 | 41 | 19.38 |
| 60 | 90 | 1.37 | 56 | 3.9 | 84 | 2.89 | 92 | 0.97 | 100 | 3.46 | 98 | 3.09 | 69 | 3.08 | 44 | 18.98 |

*Different API sources

TABLE 5

| | Media | | | | | | | | | | | | | | | |
|---|---|---|---|---|---|---|---|---|---|---|---|---|---|---|---|---|
| | pH 1.2 buffer, 900 mL, USP type II (paddle), 50 rpm | | | | | | | | | | | | | | | |
| | Batch No. | | | | | | | | | | | | | | | |
| | | | 01799-140- | | 01799-140- | | 01799-140- | | 01799-140- | | 01799-140- | | 01799-140- | | 01799-140- | |
| | | | 020116 | | 030116 | | 040116 | | 120416 | | 130416 | | 140416 | | 150416 | |
| | L0407764A1 | | Amorphous- | | Amorphous- | | Crystalline | | Crystalline | | Crystalline | | Amorphous | | Amorphous | |
| Time | RLD | | I* | | II* | | Form A | | form C | | form C | | II | | II | |
| Points | Mean | % RSD | Mean | % RSD | Mean | % RSD | Mean | % RSD | Mean | % RSD | Mean | % RSD | Mean | % RSD | Mean | % RSD |
| 0 | 0 | 0 | 0 | 0 | 0 | 0 | 0 | 0 | 0 | 0 | 0 | 0 | 0 | 0 | 0 | 0 |
| 5 | 52 | 3.71 | 39 | 25.9 | 59 | 9.78 | 27 | 25.46 | 55 | 3.33 | 95 | 3.45 | 55 | 6.07 | 49 | 17.22 |
| 10 | 59 | 2.82 | 59 | 17.23 | 75 | 9.1 | 37 | 5.09 | 64 | 2.73 | 97 | 2.28 | 68 | 5.13 | 67 | 5.43 |
| 15 | 63 | 2.55 | 67 | 14.85 | 80 | 7.02 | 45 | 5.37 | 68 | 1.49 | 97 | 2.34 | 74 | 4.92 | 76 | 5.21 |
| 30 | 66 | 2.53 | 78 | 9.45 | 86 | 7.26 | 56 | 3.74 | 72 | 1.89 | 98 | 2.46 | 82 | 4.59 | 89 | 4.01 |
| 45 | 68 | 2.19 | 82 | 7.66 | 89 | 6.56 | 62 | 3.09 | 73 | 1.87 | 99 | 2.63 | 85 | 4.38 | 94 | 3.39 |
| 60 | 69 | 2.24 | 83 | 6.73 | 90 | 5.94 | 65 | 2.77 | 75 | 3 | 99 | 2.69 | 87 | 3.76 | 95 | 2.82 |
| 90 | 71 | 1.73 | 85 | 6.02 | 92 | 4.64 | 68 | 3.2 | 75 | 1.84 | 99 | 2.21 | 90 | 3.64 | 96 | 3.06 |
| 120 | 73 | 1.33 | 85 | 5.57 | 92 | 3.15 | 72 | 7.95 | 77 | 1.96 | 99 | 2.68 | 90 | 3.17 | 96 | 2.58 |

*Different API sources

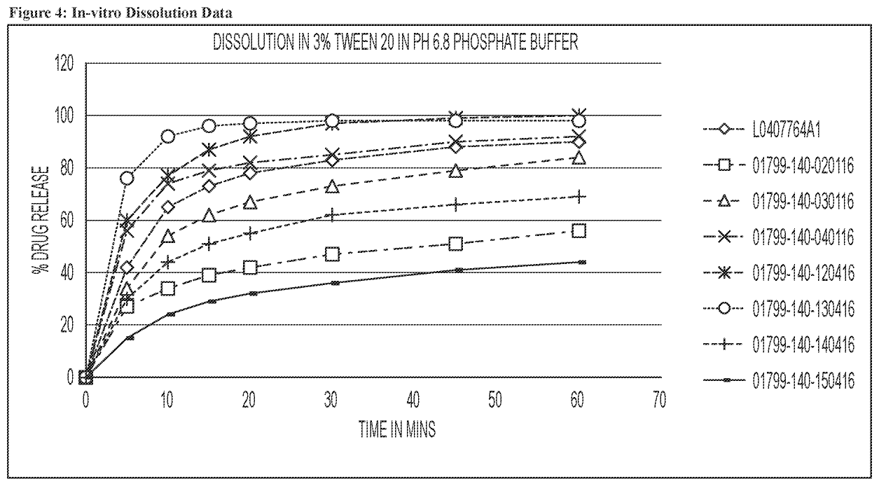

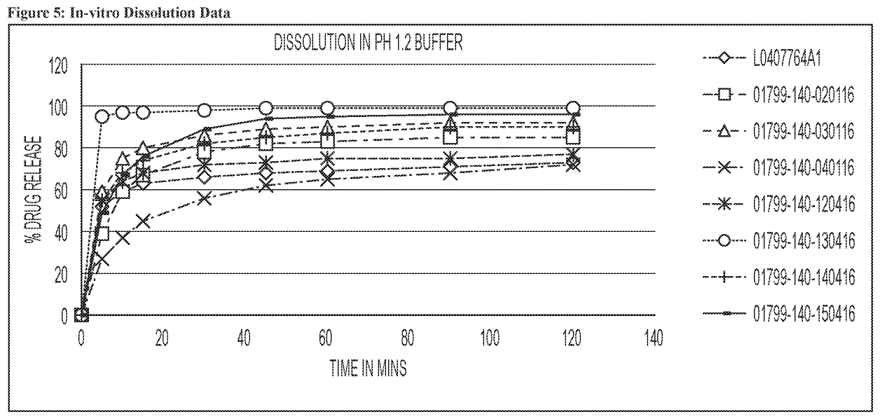

FIG. 4 shows the improved dissolution of the compositions of the invention compared to the dissolution of the capsule formulation (RLD-L0407764A1) in 3% Tween 20 with pH 6.8 phosphate buffer, 900 mL, USP type II at paddle speed of 75 rpm. FIG. 5 illustrates comparative dissolution between the capsule formulation (RLD-L0407764A1) and the compositions of the invention at pH of 1.2 buffer, 900 mL, USP type II apparatus at a paddle speed of 50 rpm.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a.” “an” and “the” include plural references unless the context clearly dictates otherwise.

1-6. (canceled)

7. A method of treating cancer by administering to a patient in need thereof a composition comprising:
a) 140 mg of ibrutinib, wherein the ibrutinib is polymorphic Form C;
b) a disintegrant in an amount from 3-30% w/w;
c) a diluent in an amount from 15-60% w/w; and
d) a lubricant in an amount from 0.1-5% w/w,
wherein the composition does not contain a surfactant.

8. A method of treating cancer according to claim 7 wherein the cancer is mantle cell lymphoma.

9. A method of treating cancer according to claim 7 wherein the cancer is chronic lymphocytic leukemia.

10. A method of treating cancer according to claim 7 wherein the cancer is small lymphocytic lymphoma.

11. A method of treating cancer according to claim 7 wherein the cancer is Waldenstrom's macroglobulinemia.

12. A method of treating cancer according to claim 7 wherein the cancer is marginal zone lymphoma.

13. The method according to claim 7, wherein the granules further comprise a coating in an amount from 1-3% w/w.

14. The method according to claim 7, wherein the disintegrant comprises croscarmellose sodium.

15. The method according to claim 7, wherein the diluent comprises microcrystalline cellulose.

16. The method according to claim 7, wherein the lubricant comprises magnesium stearate.

17. The method according to claim 7, wherein the coating comprises microcrystalline cellulose and magnesium stearate.

18. The method according to claim 7, wherein the diluent is present in an amount from 135.4-221 mg.

19. The method according to claim 7, wherein the disintegrant is present in an amount from 20.5-23.1 mg.

20. The method according to claim 7, the dosage form does not include an enteric coating.

21. The method according to claim 7, wherein oral administration of a single composition produces a total plasma concentration of at least 200 hr*ng/ml.

22. The method according to claim 7, wherein the composition is administered a single time a day.
